(19) United States
(12) Patent Application Publication
(10) Pub. No.: US 2022/0142906 A1
(43) Pub. Date: May 12, 2022

(54) COSMETIC COMPOSITION BASED ON A PLANT PRODUCT

(71) Applicant: FREEDGE SAS, SAINT-CLOUD (FR)

(72) Inventors: Laurence CAISEY, THIAIS (FR); Magali CABANNES, CLERMONT FERRAND (FR); Lucille POULET, CLERMONT FERRAND (FR); Jean-Yves BERTHON, ROMAGNAT (FR); Fabrice GUYON, SAINT-CLOUD (FR); Arnaud MARTIN, DIJON (FR)

(21) Appl. No.: 17/433,819

(22) PCT Filed: Feb. 26, 2020
(86) PCT No.: PCT/FR2020/000047

§ 371 Date: Aug. 25, 2021

(30) Foreign Application Priority Data
Feb. 26, 2019 (FR) 1901973

(51) Int. Cl. A61K 8/97; A61K 8/73; A61K 8/44; A61Q 19/00

(52) U.S. Cl. CPC A61K 8/97; A61K 8/73; A61K 2800/48; A61Q 19/00; A61K 8/44

(57) ABSTRACT

The invention relates to a composition which contains at least one plant product and at least one texturing agent, to a kit for manufacturing a cosmetic composition containing these ingredients, and to a method for manufacturing a cosmetic composition and the use of said cosmetic composition to purify, hydrate and/or enhance the radiance of the skin.

### FIELD OF THE INVENTION

The present invention relates to a composition which contains at least one plant product and at least one texturing agent, to a kit for manufacturing a cosmetic composition containing these ingredients, and to a method for manufacturing a cosmetic composition, and the use of said cosmetic composition to purify, hydrate and/or enhance the radiance of the skin.

Today, whether in the food sector or the cosmetics sector, special attention is paid to the use of natural products which are beneficial to the body and the environment. Cosmetic formulations which claim to be guaranteed to be natural have therefore appeared.

In reality, the majority of these formulations are not for the most part 100% natural since they contain synthetic preservatives which are allowed in many reference documents that certify naturalness. These formulations can also comprise allergens and are often composed of palm oil derivatives, the production of which has a very significant impact on the environment. These formulations conventionally draw their activity from plant, fruit or vegetable extracts which are present in often limited concentrations and have undergone a number of transformations which may in particular include aggressive steps for certain molecules contained in said plants, fruits and vegetables. These extracts may also be in the form of water obtained by distillation of the plants, fruits or vegetables, which is no longer a guarantee of the freshness of the fruits, plants or vegetables used or of the activity of the molecules which were present initially. These formulations are sold having long storage periods and broad storage conditions which cannot guarantee the absolute freshness of the active ingredients despite all the tests which are performed. Finally, these compositions contain mostly water which most often is not the source of any active ingredient in the composition.

There is therefore a need for new cosmetic compositions which exhibit a cosmetic activity at least as high as the compositions based on synthetic ingredients, while actually being 100% natural, preserving the environment and respecting sensitive skin.

Today, the applicant is innovative by proposing a cosmetic composition based on plant product(s) and 100% natural ingredients without synthetic preservatives, without palm oil, without palm ingredients, and without allergens to purify, hydrate and/or enhance the radiance of the skin, as well as a kit for manufacturing such a cosmetic composition, for example by the user themselves based on freshly pressed fruit or vegetable juice(s), and a method for manufacturing such a cosmetic composition.

According to a first aspect, the invention comprises a cosmetic composition comprising a plant product and at least one texturing agent.

Plant product is in particular understood according to the invention to mean the juice, the pulp or even the puree which come from a plant species.

Pulp is understood according to the invention to mean the residues of the fleshy part of certain vegetables and/or fruits.

Puree is understood according to the invention to mean crushed and/or ground fruits or vegetables.

Plant juice is understood according to the invention to mean in particular the juice of plants, preferably fruit juice or vegetable juice from plants. Vegetable juice is preferably understood to mean juice of fruit vegetables, root vegetables, leaf vegetables or stem vegetables.

The fruit vegetable designation comprises in particular eggplant, avocado, chayote, pumpkin, cucumber, squash, chili, pepper, tomato, pickle, zucchini, green bean, snow pea, papaya and/or olive.

The root vegetable designation comprises in particular beet, carrot, celeriac, chervil root, skirret, kohlrabi, galangal, ginger, ginseng, greater burdock, woodland sunflower, yam, Alexanders, cassava, turnip, parsnip, sweet potato, parsley, potato, radish, horseradish, rutabaga, purple salsify, Spanish salsify, and/or Jerusalem artichoke.

The leaf vegetable designation comprises in particular aloe vera, salad leaves, cabbage, spinach, orache, asparagus, Swiss chard, cardoon, ribbed celery, chervil, chives, lamb's quarters, bay leaf, mint, sorrel, parsley, savory, sage, thyme. It is most often the pulp of the fresh leaf of a plant.

The stem vegetable designation comprises in particular sugar cane, sorghum, tubers such as sugar beet, agave, celery, fennel, fiddleheads, rhubarb, and/or Swiss chard.

Preferably, according to the invention, the cosmetic composition comprises at least one plant product, preferably at least two plant products which are preferably fresh and unprocessed, and at least one texturing agent, selected from carrageenan, propanediol, xanthan gum, acacia gum, pectin, lecithin, glyceryl stearate, hydroxy stearyl alcohol, hydroxy stearyl glucoside, corn gum, starch, guar gum, sodium carrageenan, coconut alkanes, coconut esters, olive oil polyglycerol-6 esters, phospholipids, polyglyceryl-3 pentaolivate, and/or glyceryl citrate/lactate/linoleate/oleate.

Preferably, according to the invention, said plant product(s) is/are the juice and/or the pulp.

Preferably, said plant pulp(s) is/are selected from clementine, orange, apricot, peach, pineapple, grapefruit, lemon, tomato, currant, rhubarb, strawberry, kiwi, pear, apple, melon, grape, carrot, early carrot, pumpkin, aloe vera and/or cucumber.

Preferably, said plant juices are selected from the juices of fruit and/or the juices of vegetables.

Even more preferably, said vegetable juices are selected from the juices of fruit vegetables, the juices of root vegetables, the juices of leaf vegetables and/or the juices of stem vegetables.

“Unprocessed” is understood according to the invention to mean that said fruits and/or vegetables are used in their native form without undergoing a transformation such as heating, pasteurization, freezing or concentration before the preparation of the composition.

Indeed, fruits and vegetables contain a number of cosmetically active molecules, including natural vitamins and antioxidants, which will therefore be found in the cosmetic compositions according to the present invention, especially since said fruit or vegetable product(s) will not have been processed, these molecules being fragile and being capable of being degraded by the transformation methods.

“Fresh fruit or vegetable juice” is understood according to the invention to mean a juice obtained from a fresh fruit or vegetable without the addition of sugar or preservative. This type of juice most often has to be kept cool and has a limited shelf life. The cosmetic composition according to the invention will therefore be manufactured with this type of juice at the latest 24 hours after the juice has been obtained. The vitamins and minerals of the fruit and vegetables are particularly well preserved in this type of juice.

Preferably, said vegetable juices according to the invention are fresh and/or unprocessed.

The use of these fresh and unprocessed products therefore requires a rapid design time for the composition according to the invention in order to preserve the vitamins, antioxidants and minerals of the fruits and vegetables that are particularly well preserved in this type of juice and not degraded by the transformation methods, unlike processed products which are not fresh. Due to this rapid design, the vitamins, antioxidants and natural minerals will therefore be found in the cosmetic compositions according to the present invention which therefore differ in terms of benefit for the user from other cosmetic compositions using processed products which are not fresh.

This rapid design time is at most a week after the fresh and unprocessed fruit juice has been produced, preferably at most 24 hours after, even more preferably at most 1 hour after.

“Texturing agent” is understood to mean a compound making it possible to provide a cosmetic product with the desired texture such as a gelling agent, an emollient, or an emulsifier.

Preferably, according to the invention, the texturing agent is a gelling agent and more preferably is a natural gelling agent.

Said gelling agents which may be used according to the invention comprise, but are not limited to:
Genuvisco Carrageenan CG-131 (CP Kelco): Iota carrageenan.
Solagum AX (Seppic): Composition comprising 55 wt. % acacia gum and 45 wt. % xanthan gum
Carbogreen E. A (Azelis): Mixture of corn gum, starch, guar gum and sodium carrageenan
Genuvisco Carrageenan CG-130 (CP Kelco): Carrageenan
Uniqsens SFE (CP Kelco): Mixture of pectin, xanthan gum, and carrageenan.

Corn gum is understood according to the invention to mean a natural plant gelling agent which comes in particular from the Zea Mays species.

Starch is understood according to the invention to mean a gelling agent extracted from legumes or cereals such as corn, rice, barley or even wheat.

Guar gum is understood according to the invention to mean a gelling agent obtained by grinding the seeds of Cyamopsis tetragonolobus.

Carrageenan is understood according to the invention to mean a natural plant gelling agent which comes from extracts of red seaweed.

Sodium carrageenan is understood according to the invention to mean the salt of the natural plant gelling agent which comes from red seaweed.

Xanthan gum is understood according to the invention to mean the thickening and gelling agent obtained from the action of the bacterium Xanthomonas campestris.

Acacia gum is understood according to the invention to mean the sap exudate produced naturally by the acacia tree and having stabilizing and holding properties for cosmetic compositions.

Pectin is understood according to the invention to mean plant polysaccharides having binding and stabilizing properties of cosmetic compositions.

Preferably, according to the invention, the texturing agent is an emulsifier and more preferably is a natural emulsifier.

Said emulsifiers which may be used according to the invention comprise, but are not limited to:
Emulmetik 300 IP (Lucas Meyer): Lecithin
Simulgreen 18:2 (Seppic): Natural plant emulsifier mixing hydroxy stearyl alcohol & hydroxy stearyl glucoside and also having emollient properties.
SustOleo GMS (Inolex): Natural biodegradable glyceryl stearate without palm oil used as an emulsifier and also having emollient properties.
Olivatis 19: Olive oil polyglyceryl-6 esters and phospholipids having emulsifying properties and being 100% natural.
Olivatis 12: Polyglyceryl-3 pentaolivate having emulsifying properties and being 100% natural.
Imwitor 375: Glyceryl citrate/lactate/linoleate/oleate having a structure similar to lecithin, used for its emulsifying properties and being 100% natural.

Lecithin, or phosphatidylcholine, is understood according to the invention to mean a plant emulsifier.

Hydroxy stearyl alcohol is understood according to the invention to mean a plant emulsifying agent.

Hydroxy stearyl glucoside is understood according to the invention to mean a plant emulsifying agent.

Olive oil polyglyceryl-6 esters is understood according to the invention to mean a plant emulsifying and emollient agent.

Phospholipids are understood to mean compounds which come from membranes of plant cells for their emulsifying property.

Polyglyceryl-3 pentaolivate is understood according to the invention to mean a plant emulsifying agent.

Glyceryl citrate/lactate/linoleate/oleate is understood according to the invention to mean a plant emulsifying agent.

Glyceryl stearate is understood according to the invention to mean the fatty acid ester obtained from plant oils and having emulsifying properties.

Preferably, according to the invention, the texturing agent is an emollient and more preferably is a natural emollient.

Said emollients which may be used according to the invention comprise, but are not limited to:
Simulgreen 18:2 (Seppic): Natural plant emulsifier mixing hydroxy stearyl alcohol & hydroxy stearyl glucoside and also having emollient properties.
SustOleo GMS (Inolex): Natural biodegradable glyceryl stearate without palm oil used as an emulsifier and also having emollient properties.
Vegelight 1214 LC (Biosynthis): Coconut alkanes and coco caprylate/caprate. It is a natural alternative to volatile silicones such as cyclomethicone D5/D6.
Vegelight 1820 (Biosynthis): Coconut alkanes or C18-21 alkane. These are branched medium-chain alkanes which are natural alternatives to dimethicone.

Coconut alkanes are understood according to the invention to mean saturated hydrocarbons which come from the coconut and have emollient properties. These include in particular C18-21 alkanes (mixture of alkanes having 18 to 21 carbons and having emollient properties).

Coconut esters are understood in particular to mean coco-caprylate/caprate which is an esterified oil having emollient properties.

Preferably, according to the invention, said composition comprises Maranta powder, preferably between 0.1 and 3 wt. % of the composition.

Maranta powder or arrowroot powder is understood according to the invention to mean the powder which comes from the Maranta arundinacea plant and makes it possible to bind or thicken the cosmetic preparations while providing a non-sticky feel.

Preferably, according to the invention, said composition comprises tocopherol, preferably between 0.1 and 0.5 wt. % of the composition.

Tocopherol is understood according to the invention to mean vitamin E which is known in particular for its antioxidant properties.

Preferably, according to the invention, said composition comprises a pH regulator, preferably selected from lysine, calcium carbonate, citric acid and/or lemon juice, even more preferably between 0.1 and 2 wt. % of the final composition.

pH regulator is understood to mean an element which makes it possible to adjust (reduce or increase) the pH of the aqueous phase to approximately 5 to 5.5. Lysine, calcium carbonate, citric acid and/or lemon juice are used here as the pH regulator.

Preferably, according to the invention, said plant juice(s) is/are selected from clementine, orange, apricot, peach, pineapple, grapefruit, lemon, tomato, redcurrant, rhubarb, strawberry, kiwi, pear, apple, melon, grape, carrot, early carrot, pumpkin, aloe vera and/or cucumber juice.

Preferably, the compositions according to the invention comprise the mixture of at least two vegetable juices, and more preferably the mixtures are:
clementine—carrot—orange
carrot—orange
early carrot—apricot—peach
carrot—apricot—peach
grapefruit—lemon—pumpkin
grapefruit—pumpkin
grapefruit—rhubarb
grapefruit—rhubarb—strawberry
grapefruit—tomato—redcurrant—rhubarb—strawberry
grapefruit—lemon—tomato—rhubarb—strawberry
grapefruit—lemon—tomato—rhubarb—pumpkin
kiwi—pear—aloe vera—apple
pear—aloe vera—apple
aloe vera—cucumber
aloe vera—cucumber—melon
pear—aloe vera—apple—cucumber—melon
pear—aloe vera—apple—grape
grapefruit—tomato—strawberry
peach—orange—pineapple
grape—melon—cucumber
tomato—strawberry
apple—grape
orange—pineapple

More preferably, according to the invention, said mixtures of at least two plant juices are made in the following way:
⅓ by weight of the mixture of peach juice, ⅓ by weight of the mixture of orange juice, and ⅓ by weight of the mixture of pineapple juice, or
37.5 wt. % of the mixture of grape juice, 37.5 wt. % of the mixture of melon juice, and 25 wt. % of the mixture of cucumber juice, or
50 wt. % of the mixture of grapefruit juice, 20 wt. % of the mixture of tomato juice, and 30 wt. % of the mixture of strawberry juice, or
½ by weight of the mixture of orange juice, ½ by weight of the mixture of pineapple juice, or
70 wt. % of the mixture of pineapple juice, 30 wt. % of the mixture of tomato juice.

A person skilled in the art, a cosmetic formulator, will ensure, due to their general knowledge, that plants and in particular fruit(s) and/or vegetable(s) which make it possible to obtain a product having a sufficient amount of water, without added water, in order to make it possible to manufacture a cosmetic composition according to the invention and with the desired galenic characteristics are selected.

Preferably, according to the invention, said mixed plants are selected according to their harvest time. The plant product(s) which is/are preferably fresh and/or unprocessed and has/have the smallest possible impact on the environment will preferably be selected as far as possible from seasonal fruits and/or vegetables, i.e., which grow naturally in the manufacturing region and during the month of manufacturing of said cosmetic composition.

Preferably, according to the invention, said plant product(s) provides/provide all of the water necessary to obtain the final composition.

Preferably, according to the invention, the composition comprises 90 wt. %, more preferably 95 wt. %, even more preferably 99 wt. %, particularly preferably 100 wt. %, natural components.

Preferably, according to the invention, said compositions do not comprise palm ingredients. In particular, said compositions do not comprise palm oil.

Preferably, according to the invention, the composition obtained is in the form of a gel, serum, mask, lotion, cream, pomade, ointment, gel mask or oil-in-water or water-in-oil emulsions.

Preferably, according to the invention, said composition comprises:

| Phase | Commercial name | INCI Name | Company | Role | % |
|---|---|---|---|---|---|
| A | PINEAPPLE, TOMATO, | — | GREENTECH | — | TO MAKE |
| | STRAWBERRY JUICE | | | | UP TO 100 |
| | (PROPORTIONS: | | | | |
| | 50% GRAPEFRUIT, | | | | |
| | 20% TOMATO, | | | | |
| | 30% STRAWBERRY) | | | | |
| | PROPANEDIOL | PROPANEDIOL | CONNECT | SOLVENT | 12-18 |
| | | | CHEMICAL | | |
| | LYSINE | LYSINE | EMEISHAN | pH | 0.1-2 |
| | | | LONGTENG | REGULATOR | |
| | | | BIOTECHNOLOGY | | |
| | | | Co., Ltd | | |
| | UNIQSENS SFE | XANTHAM GUM AND | CP KELCO | GELLING | 0.1-2 |
| | SYSTEM | PECTIN AND | | AGENT | |
| | | CARRAGEENAN | | | |
| | GENUVSIC_{[DM1]}O | CARRAGEENAN | CP KELCO | GELLING | 0.1-2 |
| | | | | AGENT | |
| | CARRAGEENAN | | | | |
| | CG - 131 | | | | |
| B | SUSTOLEO GMS | GLYCERYL STEARATE | INOLEX | CO- | 1-5 |
| | | | | EMUSLIFIER | |
| | SIMULGREEN | HYDROXY STEARYL | SEPPIC | EMULSIFIER | 1-5 |
| | 18:2 | ALCOHOL (AND) | | | |
| | | HYDROXY STEARYL | | | |
| | | GLUCOSIDE | | | |
| | VEGELIGHT | COCONUT ALKANES | BIOSYNTHIS | EMOLLIENT, | 2-8 |
| | 1214 LC | (AND) COCO- | | SILICONE | |
| | | CAPRYLATE/CAPRATE | | LIKE | |
| | VEGELIGHT | COCONUT ALKANES | BIOSYNTHIS | EMOLLIENT, | 2-8 |
| | 1820 | | | SILICONE | |
| | | | | LIKE | |
| | ORGANIC MARANTA | MARANTA | GREENTECH | MATTIFYING | 0.5-3 |
| | POWDER (911009) | ARUNDINACEA ROOT | | AGENT | |
| | | POWDER | | | |
| C | COVIOX T70C | TOCOPHEROL | BASF | ANTIOXIDANT | 0.1-0.5 |

Preferably, according to the invention, said composition comprises:

| Phase | Commercial name | INCI Name | Company | Role | % |
|---|---|---|---|---|---|
| A | GRAPE, MELON, | — | GREENTECH | — | TO MAKE |
| | CUCUMBER JUICE | | | | UP TO 100 |
| | (PROPORTIONS: | | | | |
| | 25% CUCUMBER, | | | | |
| | 37.5% GRAPE, | | | | |
| | 37.5% MELON) | | | | |
| | PROPANEDIOL | PROPANEDIOL | CONNECT | SOLVENT | 12-18 |
| | | | CHEMICAL | | |
| | LYSINE | LYSINE | EMEISHAN | pH REGULATOR | 0.1-2 |
| | | | LONGTENG | | |
| | | | BIOTECHNOLOGY | | |
| | | | Co., Ltd | | |
| | GENUVSICO | CARRAGEENAN | CP KELCO | GELLING AGENT | 0.1-1 |
| | CARRAGEENAN | GUM | | | |
| | CG-131 | | | | |
| | SOLAGUM AX | ACACA | SEPPIC | GELLING AGENT | 0.1-1 |
| | | SNEGAL GUM & | | | |
| | | XANTHAM GUM | | | |
| | EMULMETIK 300 | LECITHIN | LUCAS MEYER | GELLING | 0.1-1 |
| | IP | | | AGENT/EMULSIFYING | |
| | | | | AGENT | |
| | VEGELIGHT | COCUNT | BIOSYNTHIS | EMOLLIENT | 0.1-2 |
| | 1820 | ALKANES | | | |
| | ORGANIC | MARANTA | GREENTECH | MATTIFYING AGENT | 0.1-2 |
| | MARANTA | ARUNDINACEA | | | |
| | POWDER | ROOT POWDER | | | |
| | (911009) | | | | |

Preferably, according to the invention, said composition comprises:

| Phase | Commercial name | INCI Name | Company | Role | % |
|---|---|---|---|---|---|
| A | GRAPE, MELON, | — | GREENTECH | — | TO MAKE |
| | CUCUMBER JUICE | | | | UP TO 100 |
| | (PROPORTIONS: | | | | |
| | 25% cucumber, | | | | |
| | 37.5% melon, | | | | |
| | 37.5% grape) | | | | |
| | PROPANEDIOL | PROPANEDIOL | CONNECT | SOLVENT | 12-18 |
| | | | CHEMICAL | | |
| | CARBOGREEN E.A | ZEA MAYS (CORN) | AZELIS | GELLING | 0.5-2 |
| | | STARCH (AND) | | AGENT | |
| | | CYAMOPSIS | | | |
| | | TETRAGONOLOBA | | | |
| | | (GUAR) GUM (AND) | | | |
| | | SODIUM | | | |
| | | CARRAGEENAN | | | |
| | GENUVISCO | CARRAGEENAN | CP KELCO | GELLING | 0.2-1 |
| | CARRAGEENAN | GUM | | AGENT | |
| | CG-130 | | | | |
| | GENUVISCO | CARRAGEENAN | CP KELCO | GELLING | 0.1-1 |
| | CARRAGEENAN | GUM | | AGENT | |
| | CG-131 | | | | |

Preferably, according to the invention, said composition comprises:

| Phase | Commercial name | INCI Name | Company | Role | % |
|---|---|---|---|---|---|
| A | ORANGE JUICE | — | GREENTECH | — | TO MAKE |
| | | | | | UP TO 100 |
| | PROPANEDIOL | PROPANEDIOL | CONNECT | SOLVENT | 12-18 |
| | | | CHEMICAL | | |
| | LYSINE | LYSINE | EMEISHAN | pH REGULATOR | 0.1-2 |
| | | | LONGTENG | | |
| | | | BIOTECHNOLOGY | | |
| | | | Co., Ltd | | |
| | GENUVSICO | CARRAGEENAN | CP KELCO | GELLING | 0.1-1 |
| | CARRAGEENAN | GUM | | AGENT | |
| | CG-131 | | | | |
| | SOLAGUM AX | ACACIA SNEGAL | SEPPIC | GELLING | 0.1-1 |
| | | GUM & XANTHAN | | AGENT | |
| | | GUM | | | |
| | EMULMETIK 300 | LECITHIN | LUCAS MEYER | GELLING | 0.1-1 |
| | IP | | | AGENT/EMULSIFYING | |
| | | | | AGENT | |
| | VEGELIGHT 1820 | COCONUT | BIOSYNTHIS | Emollient | 0.1-2 |
| | | ALKANES | | | |
| | ORGANIC MARANTA | MARANTA | GREENTECH | MATTIFYING | 0.1-2 |
| | POWDER (911009) | ARUNDINACEA | | AGENT | |
| | | ROOT POWDER | | | |

Preferably, according to the invention, said composition comprises:

| Phase | Commercial name | INCI Name | Company | Role | % |
|---|---|---|---|---|---|
| A | ORANGE, | — | GREENTECH | — | TO MAKE |
| | PINEAPPLE | | | | UP TO 100 |
| | JUICE | | | | |
| | (PROPORTION: | | | | |
| | 50/50) | | | | |
| | PROPANEDIOL | PROPANEDIOL | CONNECT | SOLVENT | 12-18 |
| | | | CHEMICAL | | |
| | LYSINE | LYSINE | EMEISHAN | pH REGULATOR | 0.1-2 |
| | | | LONGTENG | | |
| | | | BIOTECHNOLOGY | | |
| | | | Co., Ltd | | |
| | GENUVSICO | CARRAGEENAN | CP KELCO | GELLING AGENT | 0.1-1 |
| | CARRAGEENAN | GUM | | | |
| | CG-131 | | | | |
| | SOLAGUM AX | ACACIA SNEGAL | SEPPIC | GELLING AGENT | 0.1-1 |
| | | GUM & | | | |
| | | XANTHAM GUM | | | |
| | EMULMETIK 300 | LECITHIN | LUCAS MEYER | GELLING | 0.1-1 |
| | IP | | | AGENT/EMULSIFYING | |
| | | | | AGENT | |
| | VEGELIGHT | COCONUT | BIOSYNTHIS | Emollient | 0.1-2 |
| | 1820 | ALKANES | | | |
| | ORGANIC | MARANTA | GREENTECH | MATTIFYING | 0.1-2 |
| | MARANTA | ARUNDINACEA | | AGENT | |
| | POWDER | ROOT POWDER | | | |
| | (911009) | | | | |

Preferably, according to the invention, said composition comprises:

| | Commercial | | | | |
|---|---|---|---|---|---|
| Phase | name | INCI Name | Company | Role | % |
| A | ORANGE PULP | — | GREENTECH | — | TO MAKE |
| | | | | | UP TO 100 |
| | PROPANEDIOL | PROPANEDIOL | CONNECT | SOLVENT | 12-18 |
| | | | CHEMICAL | | |
| | LYSINE | LYSINE | EMEISHAN | pH | 0.1-2 |
| | | | LONGTENG | REGULATOR | |
| | | | BIOTECHNOLOGY | | |
| | | | Co., Ltd | | |
| | GENUVISCO | CARRAGEENAN | CP KELCO | GELLING | 0.1-1 |
| | CARRAGEENAN | GUM | | AGENT | |
| | CG-131 | | | | |

According to a second aspect, the invention consists of a method for manufacturing a cosmetic composition, comprising at least one step of mixing at least one texturing agent, selected from carrageenan, propanediol, xanthan gum, acacia gum, pectin, lecithin, glyceryl stearate, hydroxy stearyl alcohol, hydroxy stearyl glucoside, corn gum, starch, guar gum, sodium carrageenan, coconut alkanes, coconut esters, olive oil polyglycerol-6 esters, phospholipids, polyglyceryl-3 pentaolivate, and/or glyceryl citrate/lactate/linoleate/oleate, with at least one preferably fresh and unprocessed plant product.

Preferably, according to the invention, said method comprises a step of adding arrowroot powder, preferably between 0.1 and 3 wt. % of the composition.

Preferably, according to the invention, said method comprises a step of adding tocopherol, preferably between 0.1 and 0.5 wt. % of the composition.

Preferably, according to the invention, said method comprises a step of adding a pH regulator, preferably selected from lysine, calcium carbonate, citric acid and/or lemon juice, even more preferably between 0.1 and 2 wt. % of the final composition.

Preferably, according to the invention, said plant product(s) is/are obtained by pressing, centrifugation, crushing or extraction. More preferably, the step of pressing, centrifugation, crushing or extraction is carried out cold, without a heating step.

Preferably, according to the invention, once obtained, the plant product(s) is/are filtered or sieved in order to filter the pulp and to recover the juice.

Preferably, according to the invention, said plant product(s) is/are obtained by the steps of:
a. pressing, without heating, the desired elements of the plant, preferably the fruit and/or vegetables and/or roots and/or stems and/or leaves of the plant, in order to extract the juice therefrom;
b. passing said juice through a sieve in order to recover the plant products of said fruits: the juice without pulp after being passed through the sieve and the pulp remaining on the sieve.

Preferably, mixing said fresh and/or unprocessed plant product(s), obtained by the steps of pressing and passing through a sieve, with said at least one texturing agent is carried out at most one week after obtaining said plant products, preferably at most 24 hours after, even more preferably at most 1 hour after.

Preferably, according to the invention, said method comprises at least two preferably fresh and/or unprocessed plant products.

Preferably, according to the invention, if the cosmetic composition comprises at least two different fruit(s) and/or vegetable(s), said fruits or vegetables are preferably pressed and passed through a sieve separately and then the pulps obtained in this way are mixed in a step c. before being mixed with the texturing agent.

[symbol] according to the invention, if the cosmetic composition comprises at least two different fruit(s) and/or vegetable(s), said fruits or vegetables are preferably pressed and passed through a sieve separately and then the pulps obtained in this way are mixed in a step c. before being mixed with the texturing agent.

Preferably, according to the invention, said step c. may consist of a mixture of pulp with the juice.

Preferably, according to the invention, said mixing is carried out by stirring, preferably between 15 CC and 90° C., preferably between 20 and 60° C., and very particularly preferably at an ambient temperature between 20 and 30° C. Indeed, it will be particularly desirable to carry out the method at an ambient temperature in order to preserve the vitamins and minerals of the fruits and vegetables.

Preferably, according to the invention, said heating lasts between 1 and 5 minutes.

Preferably, according to the invention, said at least one texturing agent is mixed with between 30 and 95 wt. %, preferably between 65 and 85 wt. %, of the final composition of at least one preferably fresh and/or unprocessed plant product.

Preferably, according to the invention, when said composition is formulated in the form of an emulsion, said composition comprises between 60 and 95 wt. %, preferably between 65 and 82 wt. %, of the final composition of at least one preferably fresh and/or unprocessed plant product.

Preferably, according to the invention, when said composition is formulated in the form of a gel mask, said composition comprises between 45 and 90 wt. %, preferably between 68 and 83 wt. %, of the final composition of at least one preferably fresh and/or unprocessed plant product.

Preferably, according to the invention, when said composition is formulated in the form of a serum, said composition comprises 70 and 90 wt. %, preferably between 78 and 85 wt. %, of the final composition of at least one preferably fresh and/or unprocessed plant product.

Preferably, according to the invention, if the texturing agents consist of propanediol, pectin, xanthan gum, carrageenan, glyceryl stearate, hydroxy stearyl alcohol, hydroxy stearyl glucoside, coconut alkanes and coconut esters, these are mixed with between 60 and 95 wt. %, preferably between 65 and 82 wt. %, of the final composition of at least one preferably fresh and/or unprocessed plant product.

Preferably, according to the invention, said mixture comprises between 10 and 20 wt. %, preferably 15 wt. %, of the final composition of propanediol, between 0.50 and 0.75 wt. %, preferably 0.65 wt. %, of the final composition of a mixture of pectin, xanthan gum and carrageenan, between 2 and 4 wt. %, preferably 3 wt. %, of the final composition of a mixture of hydroxy stearyl alcohol and hydroxy stearyl glucoside, between 4 and 6 wt. %, preferably 5 wt. %, of the final composition of a mixture of coconut alkanes and coconut esters.

Preferably, according to the invention, if the texturing agents consist of propanediol, carrageenan, xanthan gum, lecithin and coconut alkanes, these are mixed with between 45 and 90 wt. %, preferably between 68 and 83 wt. %, of the final composition of at least one preferably fresh and/or unprocessed plant product.

Preferably, according to the invention, said mixture comprises between 10 and 20 wt. %, preferably 15 wt. %, of the final composition of propanediol, between 0.1 and 0.3 wt. %, preferably 0.2 wt. %, of the final composition of carrageenan, between 0.05 and 0.15 wt. %, preferably 0.1 wt. %, of the final composition of xanthan gum, between 0.1 and 0.3 wt. %, preferably 0.2 wt. %, of the final composition of lecithin, and between 1 and 1.6 wt. %, preferably 1.3 wt. %, of the final composition of coconut alkanes.

Preferably, according to the invention, if the texturing agents consist of propanediol, carrageenan, corn gum, starch, guar gum and sodium carrageenan, these are mixed with between 70 and 90 wt. %, preferably between 78 and 85 wt. %, of the final composition of at least one preferably fresh and/or unprocessed plant product.

Preferably, according to the invention, said mixture comprises between 10 and 20 wt. %, preferably 15 wt. %, of the final composition of propanediol, between 0.1 and 0.3 wt. %, preferably 0.2 wt. %, of the final composition of carrageenan, and between 0.05 and 0.15 wt. %, preferably 0.1 wt. %, of a mixture of corn gum, starch, guar gum and sodium carrageenan.

According to a third aspect, the invention consists of the use of the cosmetic composition according to the invention to improve the appearance of the skin and in particular to purify and/or hydrate and/or enhance the radiance of the skin after topical application.

Preferably, the cosmetic composition according to the invention is applied to the skin of the face.

According to a fourth aspect, the invention consists of a kit for manufacturing a cosmetic composition, characterized in that it comprises:
a. at least one preferably fresh and/or unprocessed plant product;
b. at least one texturing agent, selected from carrageenan, propanediol, xanthan gum, acacia gum, pectin, lecithin, glyceryl stearate, hydroxy stearyl alcohol, hydroxy stearyl glucoside, corn gum, starch, guar gum, sodium carrageenan, coconut alkanes, coconut esters, olive oil polyglycerol-6 esters, phospholipids, polyglyceryl-3 pentaolivate, and/or glyceryl citrate/lactate/linoleate/oleate;
c. optionally a pH regulator, preferably selected from lysine, calcium carbonate, citric acid and/or lemon juice;
d. optionally tocopherol;
e. optionally arrowroot powder.

Preferably, the kit according to the invention comprises a pH regulator selected from lysine, calcium carbonate, citric acid and/or lemon juice.

Preferably, the kit according to the invention comprises at least two preferably fresh and/or unprocessed plant products.

According to one embodiment of the invention, the user can press one or more fruits and/or vegetables, for example at home or at a point of sale, then mix it with a texturing agent or a combination of texturing agent and optionally a pH regulator in order to obtain a personalized cosmetic composition which is ready for use.

### EXAMPLES

### Example 1: Preparation of the Plant Products

The fruits and/or vegetables are pressed independently, without being heated, in order to extract the juice from them.

The recovered juice(s) is/are then passed through a sieve in order to filter the pulp and to recover the juice.

According to the formulations, the recovered juices or pulps are mixed in different proportions.

### Example 2: Method for Obtaining a Cosmetic Composition in the Form of an Emulsion

(Phase A)

The required amount of juice or pulp is mixed by means of gentle stirring with between 12 and 18% propanediol.

Between 0.1 and 2% lysine is added in order to increase the pH of the aqueous phase to approximately 5-5.5.

Then, while stirring,
between 0.1 and 2 wt. % of the final composition of a mixture of pectin, xanthan gum and carrageenan (Uniqsens SFE (CP Kelco)), and
between 0.1 and 2 wt. % of the final composition of carrageenan (Genuvisco Carrageenan CG-131 (CP Kelco))
   are added.

The composition is mixed until a homogenous gel is obtained.

The composition is then heated at 75° C. for 5 minutes.

(Phase B)

In a different receptacle,
between 1 and 5 wt. % of the final composition of a mixture of hydroxy stearyl alcohol & hydroxy stearyl glucoside (Simulgreen 18:2 (Seppic)),
between 1 and 5 wt. % of the final composition of glyceryl stearate (SustOleo GMS (Inolex)),
between 2 and 8 wt. % of the final composition of a mixture of coconut alkanes and esters (Vegelight 1214 LC (Biosynthis)),
between 2 and 8 wt. % of the final composition of coconut alkanes (Vegelight 1820 (Biosynthis)), and
between 0.5 and 3 wt. % of the final composition of organic arrowroot powder are mixed.

This mixture is heated at 75° C. for 2 minutes.

Phase A and phase B are then mixed by means of vigorous stirring for 5 minutes.

The mixture obtained is left to cool to 30° C. while being moderately stirred and between 0.1 and 0.5 wt. % of the final composition of tocopherol is added.

### Example 3: Method for Obtaining a Cosmetic Composition in the Form of a Serum, Hot

The required amount of juice or pulp is mixed by means of gentle stirring with between 12 and 18 wt. % of the final composition of propanediol.

Between 0.1 and 2 wt. % of the final composition of lysine is added in order to increase the pH of the aqueous phase to approximately 5-5.5.

While stirring,
between 0.1 and 2 wt. % of the final composition of lecithin (EMULMETIK 300 IP),
between 0.1 and 2 wt. % of the final composition of carrageenan (Genuvisco Carrageenan CG-131 (CP Kelco)), and
between 0.1 and 2 wt. % of the final composition of a mixture comprising 55 wt. % acacia gum and 45 wt. % xanthan gum (Solagum AX (Seppic))
   are then added.

The composition is heated at 65° C. for 5 minutes and mixed until a homogenous gel is obtained.

Then, between 0.1 and 2 wt. % of the final composition of coconut alkanes (Vegelight 1820 (Biosynthis)) is added and the composition is mixed for 5 minutes by means of vigorous stirring.

The composition is left to cool to 30° C. while being stirred.

Between 0.1 and 3 wt. % of the final composition of organic arrowroot powder is finally added.

### Example 4: Method for Obtaining a Cosmetic Composition in the Form of a Serum, Cold

The required amount of juice or pulp is mixed by means of gentle stirring with between 12 and 18 wt. % of the final composition of propanediol.

Between 0.1 and 2 wt. % of the final composition of lysine is added in order to increase the pH of the aqueous phase to approximately 5-5.5.

While stirring,
between 0.1 and 2 wt. % of the final composition of lecithin (EMULMETIK 300 IP),
between 0.1 and 2 wt. % of the final composition of carrageenan (Genuvisco Carrageenan CG-131 (CP Kelco)), and
between 0.1 and 2 wt. % of the final composition of a mixture comprising 55 wt. % acacia gum and 45 wt. % xanthan gum (Solagum AX (Seppic))
   are then added.

The composition is mixed until a homogenous gel is obtained.

Then, between 0.1 and 2 wt. % of the final composition of coconut alkanes (Vegelight 1820 (Biosynthis)) is added and the composition is mixed for 5 minutes while being vigorously stirred.

Between 0.1 and 3 wt. % of the final composition of organic arrowroot powder is finally added.

### Example 5: Method for Obtaining a Cosmetic Composition in the Form of a Gel Mask

The required amount of juice or pulp is mixed by means of gentle stirring with between 12 and 18 wt. % of the final composition of propanediol.

Between 0.1 and 2 wt. % of the final composition of lysine is added in order to increase the pH of the aqueous phase to approximately 5-5.5.

The composition is heated at 90° C. for 5 minutes while being stirred.

While stirring,
between 0.5 and 2 wt. % of the final composition of a mixture of corn gum, starch, guar gum and sodium carrageenan (Carbogreen E.A (Azelis)),
between 0.2 and 1 wt. % of the final composition of carrageenan (Genuvisco Carrageenan CG-130 (CP Kelco)), and
between 0.1 and 1 wt. % of the final composition of carrageenan (Genuvisco Carrageenan CG-131 (CP Kelco))
   are then added.

The composition is mixed by means of vigorous stirring until a homogenous mixture is obtained.

The composition is left to cool to 30° C. while being stirred.

1. Cosmetic composition which contains at least one fresh and unprocessed plant product, preferably at least two plant products, and at least one texturing agent, selected from carrageenan, propanediol, xanthan gum, acacia gum, pectin, lecithin, glyceryl stearate, hydroxy stearyl alcohol, hydroxy stearyl glucoside, corn gum, starch, guar gum, sodium carrageenan, coconut alkanes, coconut esters, olive oil polyglycerol-6 esters, phospholipids, polyglyceryl-3 pentaolivate, and/or glyceryl citrate/lactate/linoleate/oleate.

2. Composition according to claim 1, characterized in that said plant product(s) is/are the juice and/or the pulp.

3. Composition according to, claim 1 comprising a pH regulator, preferably selected from lysine, calcium carbonate, citric acid and/or lemon juice.

4. Composition according to, claim 1 characterized in that the vegetable product(s) provide(s) all of the water necessary to obtain the final composition.

5. Method for manufacturing a cosmetic composition, comprising at least one step of mixing at least one texturing agent, selected from carrageenan, propanediol, xanthan gum, acacia gum, pectin, lecithin, glyceryl stearate, hydroxy stearyl alcohol, hydroxy stearyl glucoside, corn gum, starch, guar gum, sodium carrageenan, coconut alkanes, coconut esters, olive oil polyglycerol-6 esters, phospholipids, polyglyceryl-3 pentaolivate, and/or glyceryl citrate/lactate/linoleate/oleate, with at least one fresh and unprocessed plant product.

6. Method according to claim 5, characterized in that the method comprises a step of adding a pH regulator, preferably selected from lysine, calcium carbonate, citric acid and/or lemon juice, even more preferably between 0.1 and 2 wt. % of the final composition.

7. Method according to claim 5, characterized in that said plant product(s) is/are obtained by the steps of:
a. pressing, without heating, the desired elements of the plant, preferably the fruit and/or vegetables and/or roots and/or stems and/or leaves of the plant, in order to extract the juice therefrom;
b. passing said juice through a sieve in order to recover the plant products of said fruits: the juice without pulp after being passed through the sieve and the pulp remaining on the sieve.

8. Method according to claim 5, characterized in that said at least one texturing agent is mixed with between 30 and 95 wt. %, preferably between 65 and 85 wt. %, of the final composition of at least one fresh and unprocessed plant product.

9. Use of the cosmetic composition according to claim 1 to purify and/or hydrate and/or enhance the radiance of the skin after topical application.

10. Kit for manufacturing a cosmetic composition, characterized in that it comprises:
a. at least one fresh and unprocessed plant product;
b. at least one texturing agent, selected from carrageenan, propanediol, xanthan gum, acacia gum, pectin, lecithin, glyceryl stearate, hydroxy stearyl alcohol, hydroxy stearyl glucoside, corn gum, starch, guar gum, sodium carrageenan, coconut alkanes, coconut esters, olive oil polyglycerol-6 esters, phospholipids, polyglyceryl-3 pentaolivate, and/or glyceryl citrate/lactate/linoleate/oleate;
c. optionally a pH regulator, preferably selected from lysine, calcium carbonate, citric acid and/or lemon juice;
d. optionally tocopherol;
e. optionally arrowroot powder.
